(19) United States
(12) Patent Application Publication
(10) Pub. No.: US 2022/0334033 A1
(43) Pub. Date: Oct. 20, 2022

(54) ELECTRICAL DISSOCIATION OF TISSUE SAMPLES INTO SINGLE CELLS AND/OR SMALLER GROUPS OF CELLS

(71) Applicant: BROWN UNIVERSITY, Providence, RI (US)

(72) Inventors: Anubhav Tripathi, Providence, RI (US); E. Celeste Welch, Providence, RI (US)

(21) Appl. No.: 17/721,618

(22) Filed: Apr. 15, 2022

Related U.S. Application Data
(60) Provisional application No. 63/177,211, filed on Apr. 20, 2021.

(51) Int. Cl. G01N 1/40; B01L 3/00; C12N 13/00

(52) U.S. Cl. CPC G01N 1/40; B01L 3/502715; C12N 13/00; B01L 2300/0645; G01N 2001/4038; C12N 2509/00

(57) ABSTRACT

Tissue and cellular samples can be electrically dissociated into single cells and/or smaller groups of cells. The tissue samples can be housed in a device (which may also include a fluid) with one or more electrodes residing within the device. The device can be used to process one or more tissue samples. An electric field can be established through the device and the tissue samples can be dissociated into single cells and/or smaller groups of cells under the electric field.

### CROSS-REFERENCE TO RELATED APPLICATIONS

This application claims the benefit of U.S. Provisional Application Ser. No. 63/177,211, filed 20 Apr. 2021, entitled “ELECTRICAL DISSOCIATION OF TISSUE SAMPLES INTO SINGLE CELLS AND/OR SMALLER GROUPS OF CELLS”, the entirety of which is incorporated by reference for all purposes.

### TECHNICAL FIELD

The present disclosure relates generally to tissue dissociation and, more specifically, to dissociation of tissue samples into single cells and/or smaller groups of cells by application of an electric field.

### BACKGROUND

Single-cell analysis (SCA) is a growing field that endeavors to measure the properties of individual cells. Single-cell analysis increases the resolution of cellular data while reducing background noise. In recent years, single-cell techniques have emerged as a superior analytical tool in cancer and other diagnostic applications. However, it is technically challenging to process complex tissues into viable, single cells. Existing tissue dissociation methods, such as tissue homogenization were created not for single-cell analysis, but for downstream bulk sequencing. The lack of adequate sample preparation technologies for efficient single-cell analysis from tissue poses one major limitation of the translation of SCA technology. This perpetuates the reliance on bulk analytical approaches in which all cells are analyzed together in a single sample. Bulk sequencing approaches result in low resolution and poor detection of rare cell types. Intratumor heterogeneity is more adequately resolved by studying individual cells with SCA. Knowledge of intratumor heterogeneity can guide treatment, predict driver mutations, and inform prognosis of a cancer patient. The detection of even one rare cell can be the difference between life and death. Thus, the advancement of tissue dissociation technology is necessary in order to create a more technically feasible, clinically applicable, and accurate single-cell analysis workflow.

Traditional tissue dissociation techniques are time consuming, frequently involve numerous manual preparation steps, and can lead to sub-par results. These traditional tissue dissociation techniques often utilize a temperature-controlled chemical dissociating media and/or mechanical agitation (e.g., plate shaking, centrifugation, vortexing, etc.). Traditional protocols can take hours to perform, and often involve countless pieces of expensive instrumentation. In particular, ineffective results that are observed include inefficient dissociation, viability decline, cell-type bias, and more. Existing instruments can also be difficult to adapt to different tissue types and sizes, often mandating purchase of separate reagents or components.

### SUMMARY

As an alternative to traditional tissue dissociation techniques, electrical dissociation can be used to dissociate single cells and/or smaller groups of cells from a tissue sample through the application of a controlled electric field to the tissue sample within a liquid filled cavity. Electrical tissue dissociation utilizes a more compact instrumental setup than traditional tissue dissociation techniques, and this electrical treatment modality also reduces the length of time needed to dissociate cells. Furthermore, electrical component miniaturization easily enables process multiplexing, facilitating simultaneous dissociation of several tissue cores into single cells for downstream single-cell analysis.

A system can facilitate dissociation of ex vivo or in vitro tissue samples, cellular aggregates, or microtissues into single cells and/or smaller groups of cells by application of an electric field. The system includes a device that can hold a tissue sample (or a plurality of tissue samples). One or more electrodes can reside on at least two sides of the device and can establish an electric field therebetween through the device. The electric field through the device can cause electrical dissociation of the tissue sample into single cells and/or smaller groups of cells.

A method for dissociating cell or tissue samples into single cells and/or smaller groups of cells can include: establishing an electric field through a device holding a cell or tissue sample (or a plurality of samples); and causing the sample to dissociate into single cells and/or smaller groups of cells through electrical dissociation. The electric field can be established by one or more electrodes on one or more sides of the device.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a top view of a schematic diagram of an electrical tissue dissociation system before voltage is applied;

FIG. 2 is a top view of a schematic diagram of the electrical tissue dissociation system when a voltage is first applied;

FIG. 3 is a top view of a schematic diagram of the electrical tissue dissociation system when the electric field is dissociating the tissue;

FIG. 4 is a top view of a schematic diagram of the electrical tissue dissociation system when the tissue has been dissociated;

FIG. 5 is a process flow diagram illustrating a method for electrically dissociating tissue;

FIGS. 6 and 7 are illustrations of experimental setups to test electrical dissociation of tissue;

FIG. 8 includes illustrations of an experimental process of loading and dissociating a sample for electrical dissociation of tissue;

FIG. 9 includes illustrations of a “cuvette-on-a-chip” experimental set up for electrical dissociation of tissue in a microfluidic format allowing for simultaneous visual interrogation;

FIG. 10 includes an illustration of a multiplex device for electrical dissociation and an electrical circuit schematic of the multiplex device;

FIGS. 11 and 12 include experimental finite element models of an electrical dissociation of tissue process created using COMSOL Multiphysics software;

FIG. 13 includes pictures of dissociating tissue with a 2 mm gap and corresponding microscopy images during the electrical dissociation process;

FIGS. 14 and 15 include experimental results of the electrical dissociation of tissue process investigating cellular recovery and dissociation across various DC and oscillating square wave voltage conditions;

FIG. 16 includes morphology images taken with a membrane permeable cell stain during the electrical dissociation of tissue process in element A; element B contains images taken with a nonspecific stain (Hoechst33342) and a dead cell stain (DRAQ7) to illustrate viability;

FIG. 17 includes experimental results summarizing findings of membrane integrity and roundness as well as the viability assays;

FIG. 18 includes cell cycle progression assay imaging results of cells exposed to the electrical treatment to verify that the particular electrical conditions utilized are not disrupting cellular progression through mitosis (as is the case with other cellular electrical treatments, including “Tumor Treating Fields”);

FIG. 19 includes experimental results summarizing findings from the cell cycle progression assay imaging and spectrophotometry assay;

FIGS. 20 and 21 illustrate experimental results for cfDNA free in the solution, as well as RNA content, quality, and stress pattern expression from treated samples; and

FIG. 22 includes experimental results of the electrical dissociation of human clinical glioblastoma samples.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “tissue sample” can refer to any cellular or tissue material gathered from a human patient or an animal (an ex vivo tissue) as well as any cellular or tissue material cultured in any format (an in vitro tissue, spheroid, organoid, microtissue, cellular aggregate, etc.). A tissue sample can be, but is not limited to, a solid tissue biopsy sample (e.g., skin, muscle, bone, organ, hair, etc.) taken from a patient, a surgically removed sample, or a portion of a sample such as a cryosection. It can also be any cell or tissue cultured in the lab. A specific example of a tissue sample is a tissue section, which can refer to a piece of a tissue sample specifically intended for analysis. Samples can consist of diseased tissues and cells (e.g., cancer tissue) or healthy tissues and cells, naturally occurring tissues and cells, as well as aggregates and microtissues constructed from primary cell lines, immortalized cell lines, etc. Fresh and preserved tissues can both be used.

As used herein, the term “patient” can refer to any warm-blooded organism from which a tissue sample can be taken, including, but not limited to, a human being, a pig, a rat, a mouse, a dog, a cat, a goat, a sheep, a horse, a monkey, an ape, a rabbit, a cow, etc.

As used herein, the term “tissue dissociation” can refer to methods of isolating cells (as single cells and/or smaller groups of cells) from a tissue or cellular aggregate sample of any origin, morphology, and size. Traditional methods of tissue dissociation can include enzymatic dissociation/disaggregation (e.g., using enzymes to digest cut-up tissue pieces, thereby releasing cells from tissue), chemical dissociation (e.g., using a chemical that binds with cations to disrupt intercellular bonds), and/or mechanical dissociation (e.g., plate shaking, centrifugation, vortexing, etc.). Tissue dissociation can also be achieved by applying electric fields to a tissue sample (referred to as “electrical dissociation”). Tissue dissociation can facilitate single-cell isolation (and the terms can be used interchangeably herein), which refers to processes for isolating a single-cell or type of cell from a tissue sample for later analysis.

As used herein, the term “electric field” can refer to the physical field that surrounds an electric charge and either attracts or repels other charges in the field. Electric field is defined as a vector field that associates to each point in space a force per unit of charge exerted. Field lines are a method of representing the magnitude and direction of vectors of interacting electric fields when one or more point charge is present. Any form of applied electrical potential will also be encompassed herein under the term “electric field” including DC and AC electrical currents, different waveforms, pulses, point charges, etc.

As used herein, the term “device” can refer to something designed to hold at least a tissue sample. The device can have one or more sides and may be generally cylindrical, rectangular, triangular, or the like.

As used herein, the term “cuvette” can refer to a small container with straight sides and a circular or rectangular cross section designed to hold samples such as liquids and/or tissue samples. A cuvette is a specific type of device which can be used to hold tissue and cell samples. Other devices are also used to contain these samples, but the term “cuvette” will be used herein as an umbrella term to describe the tissue containing portion of the device.

As used herein, the terms “Finite Element Modeling”, “Finite Element Analysis”, or the like, can refer to simulations of a given physical phenomenon (e.g., fluid dynamics, wave propagation, thermal analysis, stress tests, etc.) using a numerical mathematic technique known as the Finite Element Method. Non-limiting examples of Finite Element Modeling software include COMSOL Multiphysics, MFEM, GetFEM++, SimScale, Abaqus, and CosmosWorks.

As used herein, the term “electroporation” can refer to the process of applying one or more electric field to cells in order to increase the permeability of the cells' membranes to allow chemicals, drugs, or DNA to be introduced into the cell. Electroporation works by passing thousands of volts (˜8 kV/cm) across suspended cells in an electroporation cuvette. Electroporation is avoided during the electrical dissociation process and is not the governing physical phenomena behind the observed dissociation.

As used herein, the term “fluid” can refer to a substance that has no fixed shape and yields easily to external pressure.

As used herein, the term “non-ionic liquid” can refer to liquids composed of molecules that do not dissociate into ions and have negligible conductivities, but can be polarized by an electric field. A non-ionic liquid is a type of fluid.

As used herein, the term “additive” can refer to a substance added to something (e.g., a fluid) in small quantities to improve or preserve the something.

As used herein, the terms “multiplex” and “multiplexing” can refer to the process of gathering more than one set of data from the same sample as well as the process of collectively processing numerous samples at once. Multiplexing can be used to process numerous samples with the same electrical or other conditions, or it can be used to process samples with varying electrical or other conditions.

### II. Overview

Traditionally, tissue dissociation techniques often utilize a temperature-controlled chemical dissociating media, an enzyme for digesting portions of tissue, and/or mechanical agitation (e.g., plate shaking, centrifugation, vortexing, etc.). These techniques involve long and complicated protocols that can require either several bulky instruments (e.g., temperature-controlled shakers and hot plates, centrifuges, vortexers, etc.) or an expensive customized instrument that only has one purpose in the laboratory and still yields suboptimal results (e.g., GentleMACs tissue dissociator). Additionally, cell isolation results from traditional methods are often sub-par. Sub-par results from traditional techniques can be the result of multiplexing difficulties, the length of time dissociation takes, reduced cellular viability, poor cellular recovery, and low levels of successful tissue dissociation. Microfluidic devices have also been used to improve tissue dissociation in the literature, but not in existing products. They are able to improve the disruption of cellular aggregates into individual cells by incorporating microfluidic flow against microscale objects such as pillars, silica knives, or mesh or by utilizing tailored mechanical shear forces and fluid jets within geometrically optimized microfluidic channels. However, these devices can have similar issues as traditional methods of tissue dissociation and are frequently prone to clogging, require pressure-driven flow, and utilize complicated manufacturing processes not utilizable at a commercial level.

Electrical dissociation provides an alternative to these traditional techniques, providing successful tissue dissociation with compact instrumentation in a reduced timeframe. With electrical dissociation, an electric field (below traditional electroporation limits) can be established between one or more electrodes to dissociate a tissue sample into single cells, smaller cellular aggregates and/or populations of cells. Using an electric field for tissue dissociation can significantly shorten the time required for tissue dissociation, reduce the size and expense of equipment needed to perform dissociation, enable multiplexing of several samples at once, and facilitate automated tissue dissociation for downstream single cell analysis. Electrical dissociation uses one or more electrokinetic phenomena in cells responding to the application of the electric field, such as electroosmosis, electrophoresis, dielectrophoresis, electrorotation, electroorientation, and wave propagation. Electrical dissociation may provide an automated system that allows for hands-free processing of tissue samples into single cells with consistent results in a high-throughput manner. For example, such an automated system may provide direct analysis of cells dissociated from human cancer tissues in both regional and single-cell analysis, or even improve cellular recovery from tissues for bulk analysis.

Described herein is a rapid, low cost, miniaturized tissue and cellular aggregate dissociation system and method that uses applied electric fields to dissociate biopsy tissue cores into cellular suspensions. In one example, the electrical condition for tissue dissociation may be 100 V/cm at 1 kHz square wave frequency of oscillation, which may entirely dissociate a 1 mm tissue biopsy core in 5 minutes without observable cell death, fragmentation, cell cycle disruption, or significant transcriptional stress response. In another example, a 10 V/cm 1 kHz square wave was able to dissociate glioblastoma spheroids within 1 minute without observable cell death, fragmentation, cell cycle disruption, or significant transcriptional stress response.

### III. Systems

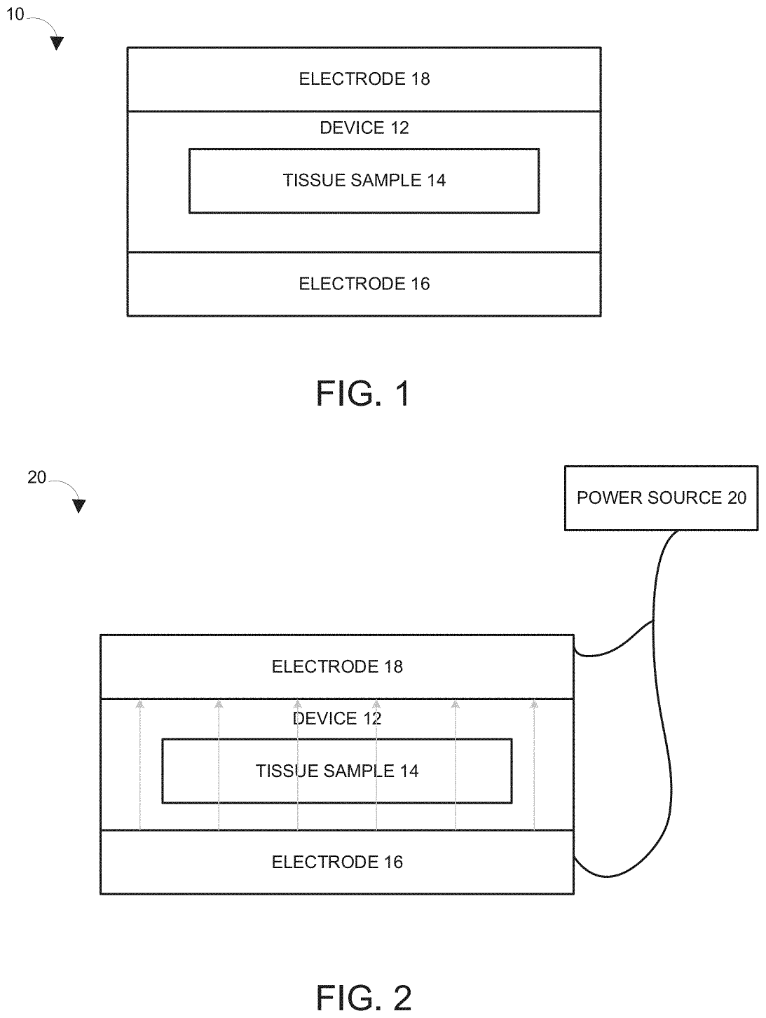

An aspect of the present disclosure can include a system 10 (FIG. 1) that can dissociate a tissue or aggregated cellular sample into single cells, smaller cellular aggregates and/or cellular populations by applying an electric field to the tissue sample. The system 10 can maintain the viability and integrity of the dissociated cells for multiplexed, downstream single cell analysis procedures.

The system 10 includes a device 12 (e.g., a cuvette) configured to hold a tissue sample 14 and one or more electrodes (shown as two electrodes 16 and 18, but it will be understood that any number of electrodes, 1 or greater, can be used) on (or within the device, near the device, etc.) at least two sides of the device 12. Illustrated in FIG. 1, the electrodes 16, 18 are on opposite sides of the device 12, but it should be understood that the electrodes 16, 18 can be on adjacent sides or the same side, or within the device 12. Additionally, electrodes 16 and 18 may also be replaced with a single electrode or three or more electrodes in implementations of the system 10. It should be understood that while the description will generally refer to two electrodes, one or more can be used in system 10. The device 12 can be configured to hold the tissue sample 14 within a fluid. The fluid can include one or more nonionic liquids and/or additives, for example. The device 12 can be configured to hold the tissue sample 14 between the electrodes 16 and 18. Ideally, the tissue sample 14 can be held equidistant or approximately equidistant from each of the electrodes 16 and 18. Alternatively, the tissue sample 14 may be held at any point between each of the electrodes 16 and 18. The system 10 shows a single device 12 configured to hold a tissue sample 12 and electrodes 16 and 18 on (or near at least two sides of the device, however it should be understood that a plurality of devices each configured to hold a tissue sample and electrodes on (or near) one or more sides of the device can be included in the system for high-throughput processing. When the system comprises a plurality of devices (each being similar to device 12) multiple tissue samples 14, which can be of the same and/or a different specimen, can be processed simultaneously. A voltage, current, and/or frequency can be specified for each of the plurality of tissue samples 14 in this example.

The electrodes 16 and 18 can establish an electric field through the device 12 to cause electrical dissociation of the tissue sample 14 into single cells. One of electrodes 16 and 18 can be a positive electrode and the other of electrodes 16 and 18 can be a negative electrode to establish an electric field between themselves when power is supplied to the electrodes. The process can also be conducted with a single electrode or with multiple electrodes. The electrical tissue dissociation can occur in a time frame of 1-15 minutes, for example in less than 15 minutes, in less than 10 minutes, or in 5 minutes or less. However, in some instances, the electrical tissue dissociation can occur over a longer time. The electrodes 16 and 18 can be, for example, plate electrodes, wire electrodes, or screenprinted electrodes. The electrodes 16 and 18 can be metal electrodes (e.g., stainless steel, aluminum, etc.) or any other type of electrode that can facilitate the generation of the required electric field (under electroporation limits). The electrodes can be planar, or have additional geometries, such as interdigitated and sawtooth.

As an example, the device 12 can be a cuvette. The cuvette can be configured to hold the tissue sample 14 in a fluid, which may include one or more nonionic liquids and/or one or more additives. The device can also be the well, wellplate, a microfluidic chip, or any other container which may hold the tissue or cell sample and/or liquid. As an example, the nonionic fluid can be ultra-pure H₂O supplemented with isotonic sucrose solution. In one nonlimiting example, the cuvette can be a 0.2 cm cuvette. The cuvette can be a horizontally positioned cuvette, a vertically positioned cuvette, a cuvette-on-a-chip, or the like. The electrodes 16 and 18 can be positioned horizontally, vertically, or at an angle relative to the cuvette (or other exemplary device 12) depending on the type of cuvette (or other exemplary device 12). A cuvette-on-a-chip can include slides covering sides of the cuvette that are orthogonal from the electrodes 16 and 18 to enable monitoring of the electrical dissociation of the tissue sample 14 under a microscope. A top slide of the cuvette-on-a-chip can be removable to load and unload the tissue sample 14. It should be understood that while two electrodes are described there can be any number of electrodes (one or more) with any electrode geometry.

As shown in the system 20 of FIG. 2, the electrodes 16 and 18 can be connected to a power source 20 by wires. For example, the wires of the electrodes 16 and 18 can be soldered, or removably attached to the device 12 and/or the electrodes 16, 18, which can be attached to the device 12, or the wires can be rested against the device 12 and/or the attached electrodes when the device 12 and electrodes are held in an insulation block, as the insulation block holds the wires in place. The power source 20 can be an adjustable electrical power supply (e.g., providing a constant voltage or current). The power source 20 can be equipped with a controlled voltage function for maintaining a uniform applied voltage (DC) or with an alternating current function (AC). The voltage can be an oscillating voltage. The output from the power source can be a linear electric field, or an oscillating electric field (e.g., square wave function, etc.). The power source can be equipped with a frequency control mechanism for oscillating the voltage applied to the electrodes 16 and 18. Other waveforms besides square wave can also be used.

The electric field generated by the electrodes 16 and 18 is shown passing from electrode 16 through the device 12 and the tissue sample 14 to electrode 18 in FIG. 2. In some instances, the device 12 can be viewed through microscope to observe the dissociation of the tissue sample 14 into single separated cells and/or smaller groups of cells. Due to the similarity of the tissue's electrical properties to the surrounding fluid and the small scale of the system 20 the electric field generated by the electrodes 16 and 18 can behave as a uniform electric field within the device 12. The voltage between the electrodes 16 and 18 is below an electroporation threshold of the tissue sample in order to maintain cellular integrity and viability during the dissociation process.

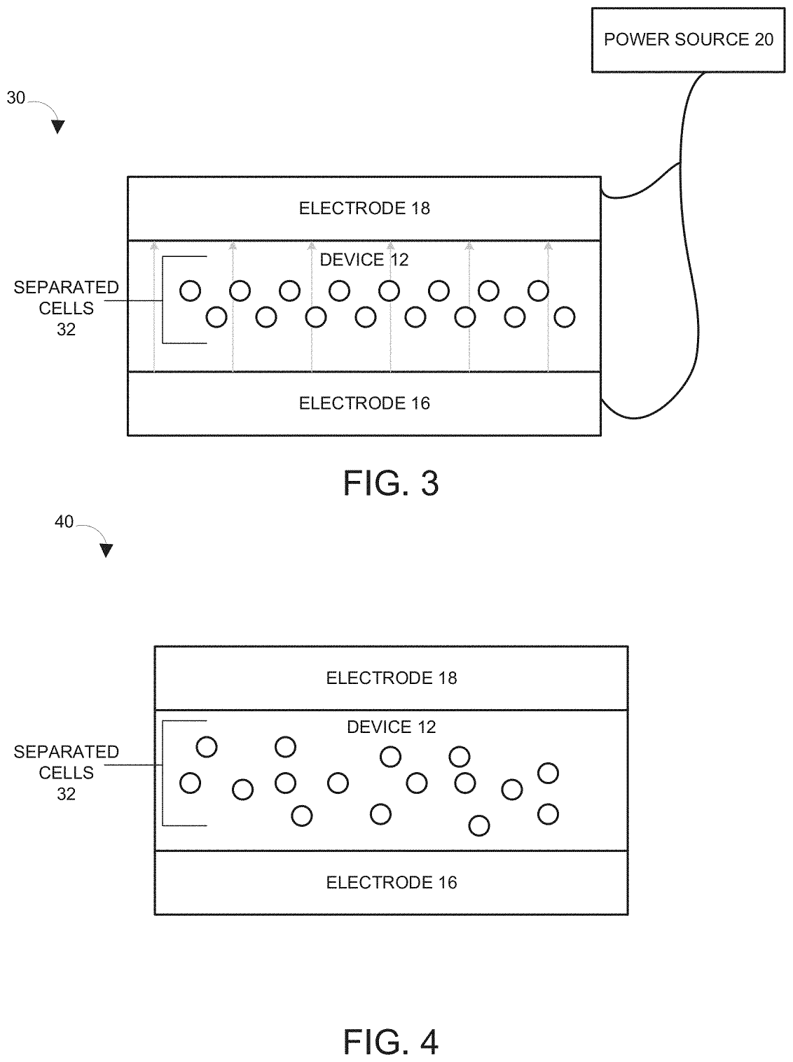

FIGS. 3 and 4 show systems 30 and 40 that illustrate the dissociation of the tissue sample in the device 12 into separate cells 32 with the application of the electric field between electrodes 16 and 18 when powered by power source 20. In system 30 of FIG. 3, the electric field causes the tissue sample 14 to break down into separated cells 32, which are suspended in the fluid inside the device 12. In system 40 of FIG. 4, the electric field between electrodes 16 and 18 has been turned off and the separated cells 32 can remain suspended in the fluid inside the device 12, but not necessarily held in place by the electric field. These dissociated cells may also settle at the bottom of the cuvette. The separated cells (or smaller groups of cells) 32 in the device 12 can be observed and quantified through a microscope and/or using imaging processing software such as ImageJ. The separated cells (or smaller groups of cells) 32 in the device 12 can also be removed from the device 12 (e.g., using a syringe, through a microchannel, or connected tubing) for multiplexed single cell analysis, for example, to determine intratumor heterogeneity. To protect against the event that one or more of the separated cells 32 remain in a clump, the systems 10, 20, 30, and/or 40 may also include at least one serpentine or other microfluidic disaggregation channel (not shown) attached to the device 12 and configured for cellular separation to separate the clumps into single cells when the clumps are flowed therethrough. In another example, the systems 10, 20, 30, and/or 40 may also include a further sample purification mechanism (not shown) that can comprise at least one microfluidic mesh removably secured between at least one input well (where the separated cells 32 can be loaded post separation) and at least one output well for collection and/or analysis of the single cells. In some instances, flowing the separated cells 32 through the microfluidic mesh can improve the purification of the single cells (e.g., to exclude any leftover debris, red blood cells, and/or off-target cells). In another example, the systems 10, 20, 30, and/or 40 may also include another type of on-device post-processing and purification mechanism to prepare purified single-cell suspensions for downstream analysis.

### IV. Methods

Another aspect of the present disclosure can include method 50 for electrically dissociating a tissue sample. The method 50 can be executed using the systems 10, 20, 30, and 40 shown in FIGS. 1-4) as well as other systems described but not pictured. For purposes of simplicity, the method 50 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 50, nor is method 50 limited to the illustrated aspects.

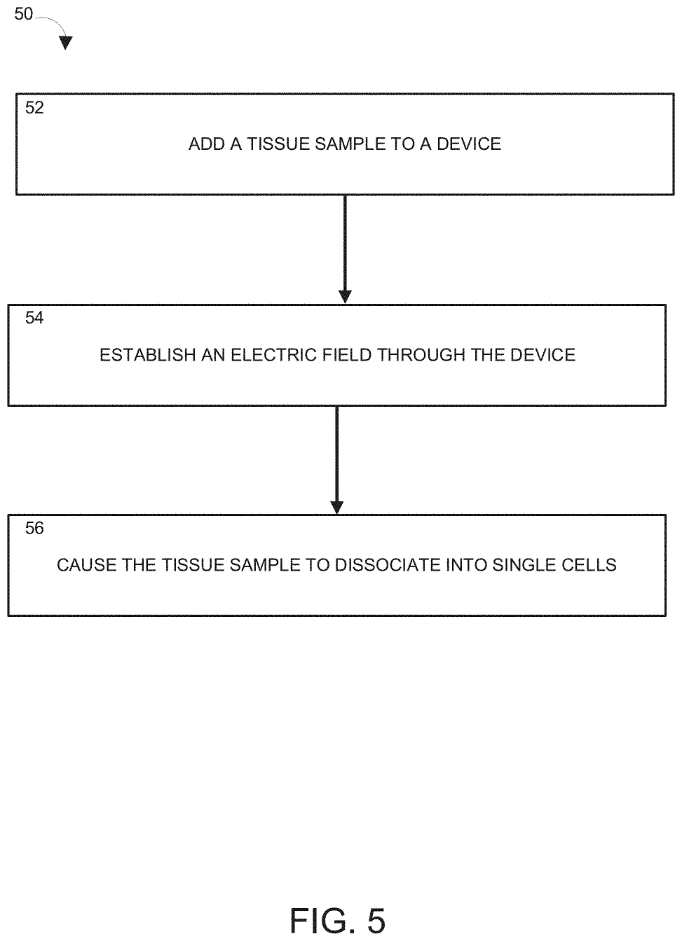

Referring now to FIG. 5 illustrated is a method 50 for electrically dissociating a tissue or cellular sample into single cells (or smaller groups of cells). At 52, a tissue or cellular sample (and in some instances a fluid) are added to a device (e.g., a cuvette, microfluidic chip, well, or other format). The tissue sample can be, for example, an ex vivo sample from a human or other mammal, or an in vitro sample of cultured primary or immortalized cells. The tissue sample can be positioned in the center of the device at an equal distance (or an approximately equal distance) from at least two opposite sides of the device, or at any location within the cavity. The device can be clear (e.g., formed out of a clear plastic, polymer, glass, or crystal) and can be configured appropriately to hold the tissue sample (in some instances, in the fluid), for example the device can be a 0.2 cm cuvette. One or more electrodes can be near or in contact with the device. If the one or more electrodes are at least two electrodes, the at least two electrodes can be positioned on one or more sides of the device—including at opposite sides (e.g., right and left sides, top and bottom sides, etc.) or adjacent sides. The electrodes can be near or adjacent to the device. The electrodes can be connected to a power source via wires that can be soldered to the device, removably attached to the device, and/or rested against the side of the device and held in place by an insulated holder. In one example, a plurality of devices, each of which can hold a tissue sample, can be included in a single system for high-throughput processing of a plurality of samples at once. The device can be a cuvette-on-a-chip, as described above, and can be positioned on a microscope following the addition of the tissue sample and the liquid. The microscope can be used to view the cuvette, tissue sample, and, in some instances, the fluid in real time during the electrical tissue dissociation. The device can consist of electrodes in any geometry, configuration, or orientation.

At 54, an electric field can be established through the device and the cell or tissue sample and, in some instances, the fluid held inside the device. The electric field can be established by the one or more electrodes positioned on one or more sides including opposing or adjacent sides of the device and attached to a power source. The power source can be an adjustable electrical direct current (DC) power supply or alternating current (AC) power supply. The power source can be equipped with a controlled voltage function for maintaining a uniform applied voltage or with an oscillation frequency function (e.g., square wave function, etc.) for oscillating the voltage applied to the electrodes 16 and 18. The power source can supply a direct current having voltages from 0 to 20 V to each of the electrodes to create electric field strengths from 0 to 100 V/cm. When the electrodes 16 and 18 are powered by an oscillating voltage, the low can be the inverse of the maximum or 0 V and the peak is the maximum. Oscillation frequencies can be from 10 Hz to 1 kHz or above. In the example where the system includes a plurality of devices a different voltage and/or frequency can be applied to each set of one or more electrodes (e.g., sequentially, at the same time, etc.).

The voltage emitted from the electrodes can be any voltage, current, and/or electric field capable of causing the tissue sample to dissociate into smaller aggregates and single cells that remains below the electroporation threshold of the tissue sample, so as to maintain cellular integrity and viability. For example, the voltage can be a DC voltage or an AC voltage (to establish a DC electric field or an AC electric field) with or without a frequency. As an example, the frequency can be greater than 1 kHz, but in some instances, the frequency can be less than 1 kHz. At 56, application of the electric fields can cause the tissue or cellular sample to dissociate into single cells and/or smaller groups of cells through electrical dissociation. The tissue or cell sample can be dissociated in time intervals of <1-15 minutes, or in time intervals exceeding 15 minutes. For example, less than 15 minutes, in less than 10 minutes, or in 5 minutes or less (or time) greater than 15 minutes. The single cells and/or smaller groups of cells can be suspended in the fluid in the device and can be visually observed and quantified (e.g., through a microscope and/or using image processing software) and/or removed from the cuvette (e.g., using a syringe, through a microchannel, or connected tubing) for downstream single-cell and other analysis. The single cells dissociated from the tissue sample can be analyzed, for example, to test intratumor heterogeneity of the tissue sample.

The method 50 can be carried out with a single cuvette, on a microfluidic chip, in a tube, or with a well plate configured with a plurality of wells for holding a tissue sample and a liquid with electrodes positioned on opposing sides of each well, amongst other formats. Additionally, one or more steps of the method 50 can be automated with a controller connected, via a wired and/or wireless connection, with at least the power source and/or the electrodes. The controller comprising a processor for executing instructions and a non-transitory memory for storing the instructions.

In order for a sample to be suitable for analyses such as single-cell sequencing, cellular suspensions must consist entirely of single-cells. In order to further dissociate any remaining cell clumps within the sample, various microfluidic and other post-processing steps can be completed within the device, or off the device. In one example, the processed sample, comprising the separated cells, can be flowed through at least one serpentine or other disaggregating microfluidic channel attached to the device to separate the clumps for further processing and analysis. In another example, the processed tissue sample, comprising the separated cells, can be flowed through at least one microfluidic mesh removably secured between at least one input well (where the separated cells can be loaded post separation) and at least one output well for collection and/or analysis of the single cells. In some instances, flowing the separated cells through the microfluidic mesh can improve the purification of the single cells (e.g., to exclude any leftover debris and cellular aggregates).

### V. Experimental

The following experiments demonstrate tissue dissociation using experimental setups of the system(s) and methods described above. Based on these experiments, cells can be dissociated from a tissue or cellular aggregate sample using an electric field.

### Methods

Electrical Parallel Plate Electrode Setup

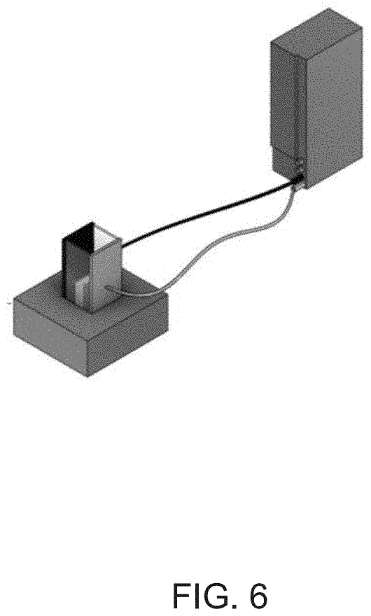

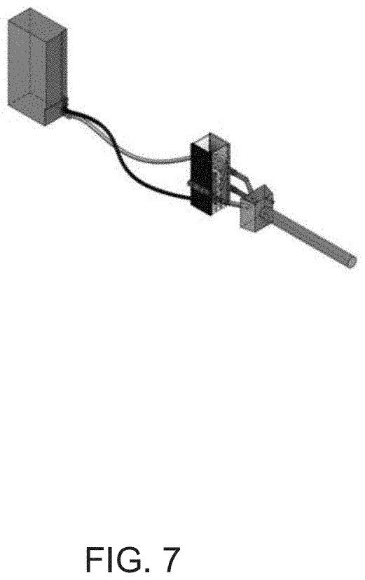

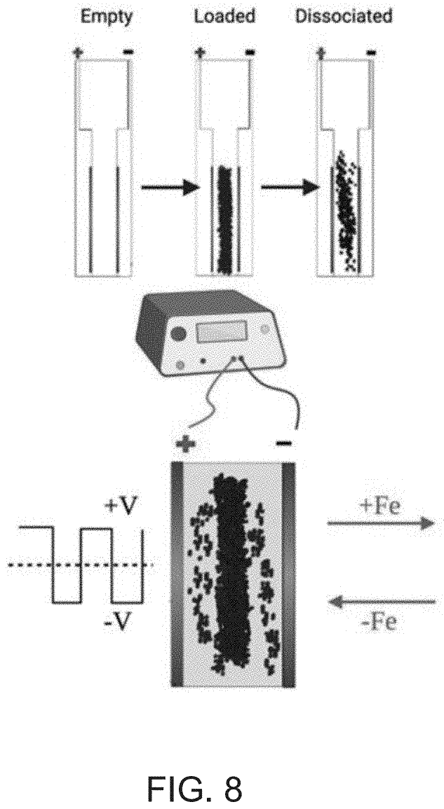

Some of the tested electrical setups (shown in FIGS. 6-8) consisted of the following components: a 0.2 cm gap length plastic encased electrode cell with two parallel plates, an adjustable electrical power supply, complete with two micro-electrodes, and a custom-made insulating holder. For oscillating voltage trials, a controlled wave function generator was used. Multimeters and oscilloscopes were used for validation of output. The power supply was equipped with a controlled voltage function, which was used to maintain a uniform applied voltage to the parallel plate electrode cell over the duration of the various experiments. FIGS. 6 and 7 illustrate the different electrical device configurations used in some of the short time course (<5 minutes) and long-time course (<30 minutes) trials, respectively. FIG. 8 shows a schematic representation of the process of electrical dissociation

Cuvette-On-a-Chip Fabrication

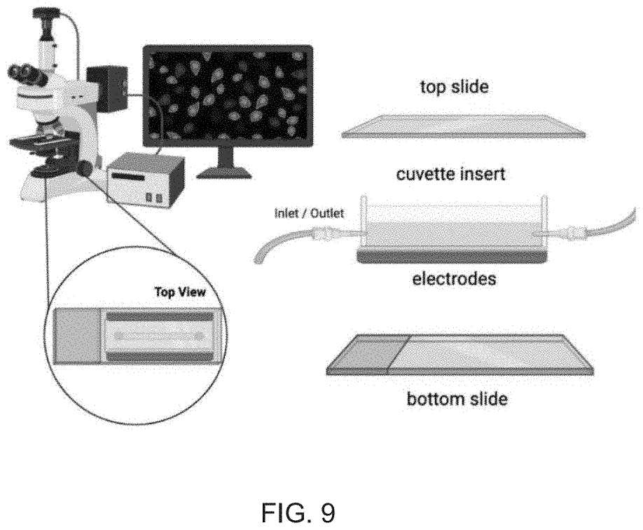

A prototype microfluidic chip, shown in FIG. 9, was created for the purpose of characterizing the phenomenon behind the electrical dissociation and progressing the dissociation processing workflow. The chip enabled viewing of the dissociation phenomenon under a microscope.

The chip was fabricated as follows: The aluminum electrodes were removed from the cuvette and placed on a custom optically transparent glass slide chip, which was itself placed into an imaging dish. Wires were attached to the sides of the electrodes in order to transmit Voltage. The original electrodes and gap length were maintained in order to limit experimental variability. The cuvette could be fitted with tubing at either end to retrieve sample more effectively via a simple pump.

After this, fluid was loaded onto the chip. The tissue biopsy was loaded into the interior of the chip and the electric field was applied through the chip via the electrodes. The dissociating tissue section could then be imaged using microscopy. At the end of a given time course, therefore, easy cellular retrieval could be facilitated by triggering the automated fluid pump, which removed the liquid and cells from the cuvette, instead of complicated sample removal from the vertical cuvette.

Arduino Generator and Multiplexed Device

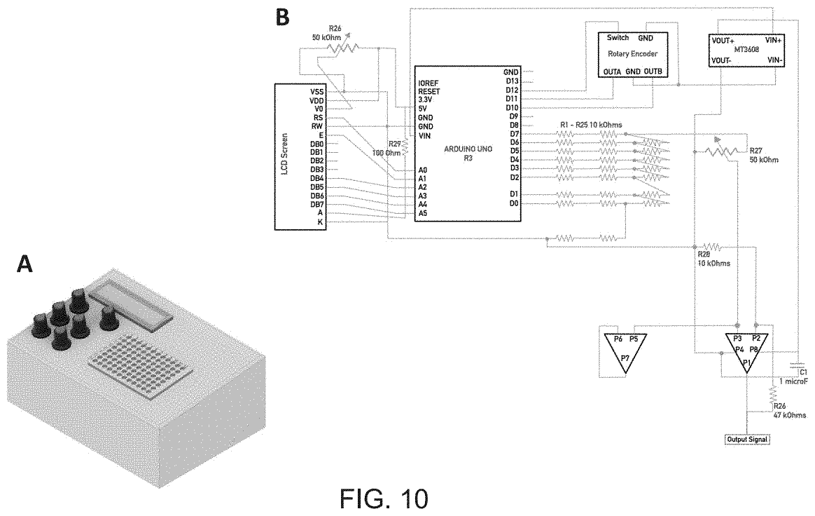

A compact, low-cost device enabling controlled electrical output programmable with different voltages, waveforms, and frequencies was created and programmed using an Arduino Uno microcontroller. A second device was created and programmed using an Arduino Due microcontroller in order to simultaneously process numerous different tissue sections with individually programmable electrical conditions. FIG. 10, element A shows an example illustration of the second device. FIG. 10, element B shows an example electrical schematic for the device.

COMSOL Multiphysics Modeling

In order to obtain a greater predictive understanding of the electric field within the parallel plate electrode cell, COMSOL Multiphysics modeling was performed using the AC/DC module. 3D model geometries were designed using two parallel plate electrodes composed of stainless steel, the 0.2 cm cavity filled with ultra-pure water, and a tissue cylinder model of the dimensions used in the study (diameter of 1 mm, height of 5 mm). Finite analysis was performed by meshing the components using free triangular mesh with a minimum element size of 0.001 cm. A grid independence study confirmed that the calculated solution was independent of the mesh size.

The boundary conditions were set by defining the edge of the left electrode as the applied voltage while the second electrode was defined as the ground. The conductivity of the tissue cylinder within the cuvette was set as 0.57 S/m, the known conductivity of healthy porcine liver as found in another study. However, it is possible that temperature fluctuation could result in increases to the conductivity of the tissue, as also observed in a liver tissue conductivity study. A slightly higher conductivity value was used to accommodate any fluctuation. The stainless-steel electrodes' conductivity was 1.45×10\hat{ }6 S/m and the conductivity of the ultra-pure water was 0.05 μS/cm, as verified using a conductivity meter. An LCR meter was used to determine the dielectric constant of the LCMS grade H2O (78.4) by measuring the capacitance between the plates.

The COMSOL results confirmed that the electric field strengths were as anticipated—for example, 10 V/cm for an applied voltage of 2 V, 100 V/cm for an applied voltage of 20 V, and so on. This provided insight into the optimum field strength for dissociation of tissue placed within that particular field. All voltages that were tested experimentally were also tested in COMSOL.

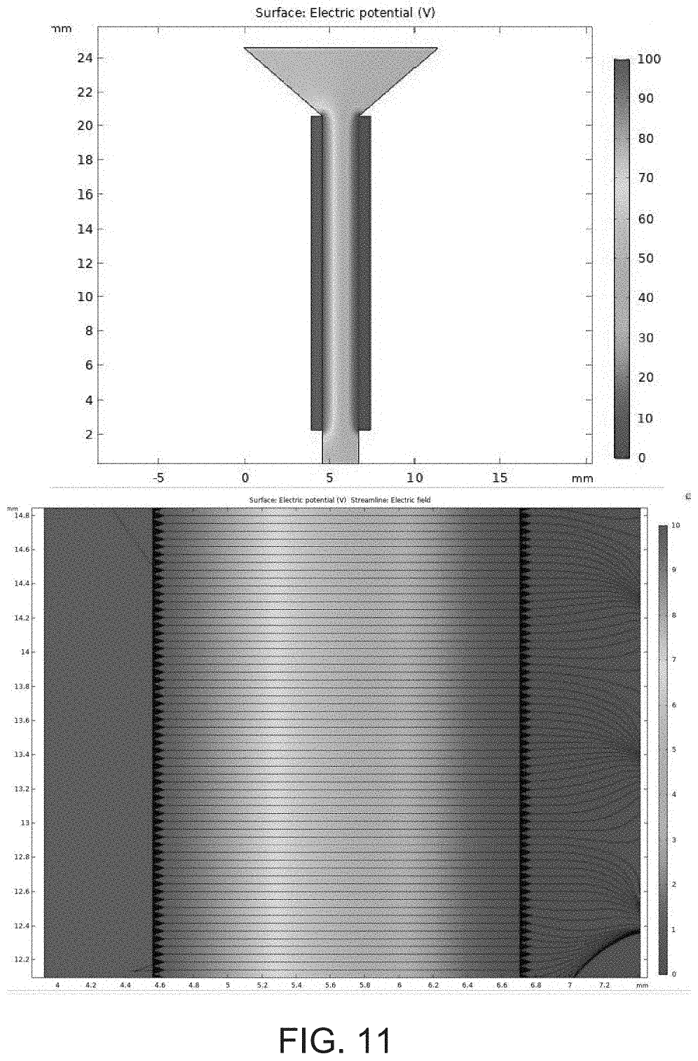

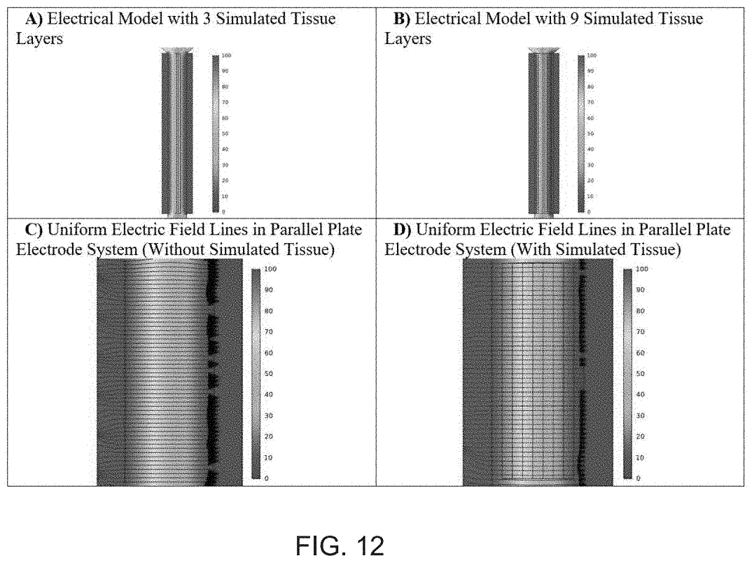

Additionally, in order to assess whether these physical results would translate to the other electrical setups, the “cuvette-on-a-chip” setup was modeled in COMSOL and tested as well. Results were consistent across all electrical setups included. FIG. 11 shows COMSOL models of the cuvette containing only water modeling the uniform (e.g., linear) electric field behavior in the cuvette (represented by field lines). Element A of FIG. 12 Illustrates the electrical model with 3 simulated tissue layers and Element B of FIG. 12 illustrates the electrical model with 9 simulated tissue layers. The tissue layers decrease in conductivity and increase in permittivity to simulate actively dissociating tissue cores. Element C of FIG. 12 shows uniform electric field lines in cuvette without simulated tissue, illustrating the linearity of the electric field across the cavity. Element D of FIG. 12 shows uniform electric field lines in cuvette with simulated tissue model, illustrating that the electric field linearity is not disrupted by the presence of tissue within the cavity.

Media Testing

When conducting the electrical experiments, a media test was first performed. In the first test, the gap in between the two metallic plates of the parallel plate electrode cell was filled with 300 μL of ultra-pure deionized water or media. Various phenomena, such as sample loss to bubbling, heating, conductivity, and pH fluctuation were then measured. This test was performed without any cells or tissue to assess liquid sample recovery in various electric field conditions for low conductivity (ultra-pure water) and high conductivity (DMEM media) solutions.

Preliminary tests examining the effect of media on cells were then conducted using aliquots of MDA-MB-231 cells that were pre-counted using a hemocytometer. The same two solutions were tested, as well as a 300 mM sucrose solution that aimed to reduce the osmotic stress burden on cells in the ultra-pure water. The cells were exposed to the three conditions without any applied electric field and were examined at 5-, 15-, and 30-minute timepoints using the live-dead staining protocol with microscopy and ImageJ analysis. Viable cell recovery could then be assessed as a percentage, enabling a deeper understanding of when cell lysis and death begin to occur in various media. These experiments were both conducted prior to the electrical tissue dissociation experiments in order to optimize the media component of the workflow.

Electrical Dissociation Protocol

Subsequent <5-minute tests were performed using 300 μL of ultra-pure filtered water, unless otherwise indicated. This design choice was informed by the negligible osmotic stress and effect on viability in <5-minute trials, as determined in the above experiments. A 1 mm diameter tissue biopsy core was taken from a bovine liver tissue specimen as described previously using a Robbins Instruments biopsy tool. The biopsy core was then loaded vertically into the cavity between the electrodes, and positioned equidistant from, but not touching the two electrodes.

In <5-minute experiments, the electrode cell was then positioned within the insulating holder, and the electrode wires were placed at either side of the device, putting them in contact with each respective metal plate (FIG. 6). The power supply, which was pre-set to a specific voltage of interest, was then turned on. Actual voltages and amperages were independently verified using a multimeter. The voltage was controlled within the experiments. and different voltages were tested, as expressed in electric field strengths of 10-100 V/cm etc. Actual voltages of 2-20 V etc. were applied to the plates in order to achieve these conditions. These electric field strengths and voltages are well below the established electroporation threshold.

At different time intervals over a period of 5 minutes, the voltage was automatically stopped and the entire 300 μL liquid sample of dissociated cells in suspension was withdrawn from the device using a sterile 20 Gauge needle. Trials were replicated at least ten times in order to verify experimental reproducibility.

For long time course <30-minute trials only, an alternative setup was developed (FIG. 7). The setup used a holding rack in order to secure the electrodes in place over time. The electrodes were secured to the parallel plates on either side of the cuvette using the rack. Electrical tape was used to ensure insulation. Water was supplemented with 300 mM sucrose.

For oscillating voltage trials, the information was programmed into the function generating power supply system, and the experiment was left to run in the same manner as the 0 Hz DC voltage experiments. A square wave function was used, in which the peak of the wave was equal to the maximum voltage and the trough was equal to the minimum voltage of equal magnitude. Various frequencies were tested, including a lower limit of 10 Hz and upper limit of 1 MHz, based on the frequency limitations of the function generator.

Immediately after all treatments, cells were pelleted so that the supernatant could be removed. They were then transferred into a solution containing PBS, in order to prevent cell lysis from hyperosmotic swelling.

Horizontally Oriented Device Fabrication

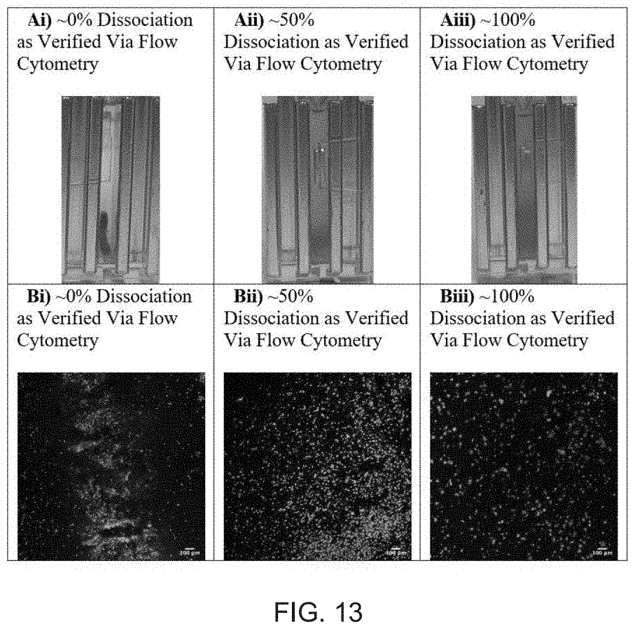

A prototype microfluidic chip (see FIG. 9) was created for the purpose of optically interrogating the phenomenon of electrical dissociation in real time. The chip enabled viewing of the dissociation phenomenon under a microscope, which was challenging in the vertical orientation (see FIG. 13, elements A sub i-sub iii and B sub i-sub iii). In FIG. 13, elements A sub i-sub iii show pictures of dissociating tissue within 2 mm gap are represented, while corresponding microscopy images taken in real time are represented in FIG. 13, elements B sub i-sub iii. FIG. 13, element A sub i and element B sub i represent a baseline of dissociation corresponding to having just submerged the tissue. A small number of surface cells are immediately washed off. FIG. 13, element A sub ii and element B sub ii represent ˜50% dissociation of the tissue, while FIG. 13, element A sub iii and element B sub iii represent ˜100% dissociation of the tissue using applied electric fields. All qualitative results presented were quantitatively validated using flow cytometry.

Tissue and Cell Sources

Bovine liver tissue was utilized in dissociation characterization tests using a previously reported protocol. The tissue was obtained from a local butcher and promptly cryopreserved for later analysis.

As live cells were needed to examine effects on viability, MDA-MB-231 triple-negative breast cancer cells were cultured for use. The MDA-MB-231 cells were cultured in media consisting of Corning DMEM with L-glutamine, 4.5 g/L glucose, and sodium pyruvate supplemented with 10% fetal bovine serum (GE Healthcare) and 1% Penicillin-Streptomycin. Partial passage was used to elute three-dimensional cellular clumps, which were tested in the dissociation workflow for the purpose of examining viability of live cells only. These cells were not used to assess ex vivo dissociation efficacy due to their limited complexity in comparison to ex vivo tissues.

Human clinical glioblastoma tissues were tested in dissociation characterization tests. Tissues were obtained immediately following tumor removal surgery and were sectioned into 1 mm pieces and subsequently processed.

Primary cells isolated from human clinical glioblastoma tumors were also tested. The cells were cultured at 5-day intervals in non-coated 60 mm dishes at a 1,000,000 cells per dish initial seeding density. The cells naturally form primary neurospheres or “spheroids” when cultured in a non-coated dish. The cells were suspended in a primary neurosphere complete medium, which consisted of 47.74 mL of 1× Neurobasal A, 0.5 mL of 2 mM GlutaMAX-I Supplement, 0.5 mL of 100× Anti-Anti, 100 μL of 20 ng/mL bFGF and EGF, 1 mL of B-27-A, and 50 μL Heparin.

Flow Cytometry

Flow cytometry was used in the bovine liver tissue experiments to assess the total number of dissociated cells across various electrical treatments and compare these results to control and chemical/mechanical treatments. Cell counting in flow cytometry was performed using a previously described size gating method developed by the inventors. This method of inferring cell size using size-controlled flow cytometry beads and cell type bins was combined with an additional layer of security. An extra step was added to the sample preparation protocol by treating the dissociated cells with red blood cell lysis buffer, DNAse I, and then staining with Hoechst 33342 to nonspecifically stain the nuclei of cells. This method enabled quantification of tissue cells while distinguishing them from cellular debris and other particles within the sample.

DNAse I Treatment

DNAse I solution was prepared by combining 327 μL of nuclease free water with 60 μL of DNAse I buffer (PerkinElmer) and 3 μL of DNAse I stored in glycerol. After preparation, the stock solutions were stored in a 4° C. refrigerator.

The cellular suspension was spun down using a centrifuge at 1,500 RPM to form a cellular pellet. The supernatant (˜300 μL ultra-pure water) was then removed with a pipette, while being careful not to disturb the pellet. The cells were treated with 20 μL of DNAse I. After the DNAse I solution was pipetted onto the cells, the cells were resuspended out of the cell pellet and into the solution via gentle agitation.

The solution was then incubated with the cells for 5 minutes, centrifuged, and removed with a pipette. After the DNAse solution was removed, the cells were then resuspended in ˜248 μL of PBS and 2 μL DNAse solution, which served as a recommended low maintenance concentration. The tube was gently agitated to evenly disperse the cells.

Hoechst 33342 Staining for Flow Cytometry

The Hoechst 33342 stain is a readymade product for flow cytometry live/dead staining purchased from ThermoFisher Scientific (Hoechst 3342 Ready Flow Reagent, Thermofisher Scientific). Instead of dropping the dye, a more quantitative approach was taken by pipetting the dye in known volumes and concentrations in order to reduce variability.

The DNAse treated cell solution was split into 2 aliquots of 125 μL used in flow cytometry analyses in replicates. The stain was placed at the recommended concentration within each sample tube. The tubes were then incubated at 37° C. for 30 minutes with the stain. Afterwards, the contents of the tubes were pipetted onto 96 well plates. The plates were filled with an equal volume of PBS to a volume of ˜250 μL, 125 μL of which was analyzed on the flow cytometer.

Mathematical Modeling of Expected Cell Numbers

Dissociation efficacy was quantified using a previously reported comprehensive methodology developed by the inventors that employs a combination of techniques in order to examine the efficacy of dissociation and cellular retrieval. Cell count estimates based on surface area and weight of bovine liver tissue specimens are synthesized into a single mathematical model which was used to calculate the percent dissociation for each sample. Prior to this work, the model was established to have a Pearson R-squared correlation value of 0.93 and 2-tailed P value <0.001 when comparing the theoretical predicted values to the experimentally obtained values.

Microscopy Viability Assay

Viability tests were performed on live, freshly passaged MDA-MB-231 triple negative breast cancer cells. The cells were exposed to the same electric field conditions as tissue sections. They were microscopically examined using both hemocytometry and fluorescence microscopy in order to assess cellular integrity and viability. Live and dead cells were quantified using the Image Processing Workflow described below and characterized in part elsewhere.

Viability Assay: DRAQ7 & Hoechst 33342 Stain

An Olympus FV3000 confocal microscope (Brown University Leduc Bioimaging Facility) was used to assess viability and membrane integrity of MDA-MB-231 cells. A chemical and mechanical control was compared to both a 100 V/cm DC electric field condition, as well as a 1 kHz oscillating voltage condition with the same electric field strength.

Hoechst 33342 was again used as a nonspecific stain, while DRAQ7 was used as a “dead” stain. An anthracycline derivative, DRAQ7 enters through cells with compromised membrane integrity, binding to DNA. It can be useful in the real-time monitoring of cell death, as it does not induce death, but serves as an effective marker of compromised membrane integrity. These two dyes were co-stained for fluorescent microscopy “live/dead” analyses, and 10 μL samples were placed onto imaging dishes for investigation at 10×, 20×, and 100× oil-immersion.

Mitotic Cell Cycle Assay

An assay for mitotic cells and cell cycle progression was conducted using an established protocol. As cell cycle disruption at the mitotic exit phase has been observed with higher frequency electric field treatments (e.g., 200 kHz), it was important to examine whether this effect occurs here.

Cellular suspensions of MDA-MB-231 were either not treated or electrically treated at 100 V/cm 1 kHz. Afterwards, Anti-phospho Histone H3 (Ser10) Antibody AlexaFluor488 Conjugate (Sigma-Aldrich) was used to stain selectively for phosphorylated Histone H3, an indicator of mitosis. A recommended 1:50 dilution of antibody was prepared in PBS, and co-incubated with cells at 37° C. for 1 hour. 10 μL samples of cells were then visualized under the confocal fluorescence microscope in imaging dishes and images taken for analysis at 10×. Cell-count matched samples were also analyzed for relative fluorescence intensity using a ThermoFisher Nanodrop 3300 fluorospectrometer.

Image Analysis Platform

The ImageJ-FIJI image analysis software was used for the purposes of cell counting from confocal microscopy images, morphology assessment, and live dead image processing (National Institutes of Health). A workflow utilized in previous work by the inventors was applied here for visual processing.

This same image analysis workflow was used both for image processing of cells stained with a single fluorescent probe, as well as images with two different fluorescent probes. Images with two different probes required a simple additional step of discerning between different fluorophores by setting fluorescence thresholds before proceeding with the cell counting workflow.

CfDNA Assay

The cfDNA in dissociated tissue samples untreated with DNase I was analyzed at 5-, 15- and 30-minute time points in an untreated control condition and various electric field oscillation frequencies to assess whether genetic contents are released from cells during the electrical treatment. All cells were removed from the 300 μL solution, leaving only the supernatant. The QIAGEN QIAamp Circulating Nucleic Acid Kit was then utilized to extract and prepare the circulating nucleic acids, and RNase digestion was performed to purify just the cfDNA. The cfDNA was subsequently quantified by dropping 1 μL onto the Nanodrop 1000 Spectrophotometer and measuring absorbance at 260 nm with respect to 280 and 230 nm.

RNA Analysis

Samples of 500,000 MDA-MB-231 cells each were exposed to either a control consisting of no treatment, an optimized chemical/mechanical treatment, or an optimized electrical treatment of 100 V/cm 1 kHz. Other samples were exposed to the optimized electrical treatment and then added to media and placed in a thermal controlled CO₂ incubator for 15 and 60 minutes, respectively, to assess the effect of a “recovery period”.

After treatment, the cells were pelleted via centrifugation and RNA was extracted from the populations using the QIAGEN RNeasy Micro Kit. DNAse I digestion and RNA cleanup were performed using the same spin column kit, following QIAGEN protocols. The RNA was eluted into 30 μL of nuclease free, RNase free water. The column was then washed with an additional 30 μL of water.

Total RNA was then quantified using the Nanodrop 1000 Spectrophotometer by dropping 1 μL of the RNA sample onto the pedestal and again measuring absorbance at 260 nm with respect to 280 and 230 nm. The Agilent 2100 BioAnalyzer was then utilized for confirming total RNA concentration and ascertaining the RNA Integrity Number (RIN) with the RNA Nano chip.

Samples were adjusted to the same RNA concentration by adding RNase free water or utilizing controlled evaporation centrifugation. Reverse transcription of RNA into cDNA was then performed using the Applied Biosystems High-Capacity cDNA Reverse Transcription Kit. qPCR was performed for 6 probes which have an established relationship to MDA-MB-231 stress-response—SERPINE1, INHBE, FLRT1, HSPA5, ECM2 and PLAT22. Custom ThermoFisher TaqMan probes were created to amplify these targets in qPCR. Expression changes were examined in the chemical/mechanical and electrical treatment groups in comparison to the untreated control-established baseline.

Statistical Analysis

All studies were performed with a minimum of 5 biological and 3 technical replicates. Results are represented as average±standard deviation. Where relevant, a one-way analysis of variance (ANOVA) or a two-way ANOVA was performed. Tukey's post-hoc test and 95% confidence interval were used for analysis. Multiple comparison analysis was used to assess relationships between variables. * p<0.05, ** p<0.01, ***p<0.001, **** p<0.0001. All statistical tests were conducted using Graph Pad Prism software.

Results

Physical Modeling of Electric Fields

In this work, a method of dissociating tissue into cells using applied electric fields was developed. Prior to experimentally investigating parameters of electrical dissociation, a physical model was created to assess whether electric-field linearity would be preserved within the parallel plate electrode cell. Tested voltages of 2-20 V were applied across the electrodes and through a simulated cavity with just water as well as water with a simulated tissue core, and water with 3-9 layers of simulated dissociating tissue (FIG. 12, elements A and B). The electric field was linear between the electrodes across all tested conditions, was not deflected by the tissue, and did not create any hot spots within the cavity (FIG. 12, elements C and D).

Effect of Media Composition on Sample Recovery

The effect of media composition on sample recovery and sample loss due to bubbling was investigated prior to conducting comprehensive tissue dissociation studies. Ultra-pure water was compared to media in order to assess relative recovery of respective liquid samples. Water was used in this context as a low-osmotic strength and low-conductivity solution, while the media, which contained several added salts and ions, represented a higher osmotic strength and conductivity solution. The Debye length of the water was approximately 10× that of the media. The media also had a notably higher viscosity. Other buffers such as 300 mM sucrose are examples of how solutions can be made isotonic to cells while retaining their low conductivity.

Although much less significant in water trials, low-level bubbling was also observed to take place in higher DC electric field strengths such as 100 V/cm, resulting in 36±11% sample losses at 5 minutes in comparison to 78±10%. Other research has shown that, below 1-10 kHz, electrolysis is frequently observed in low conductivity solutions. This may help to explain the excessive bubbling which led to low sample recovery in the 100 V/cm treatment with no oscillation, and why 1 kHz oscillation frequency showed improved results of only 8±7% losses after 5 minutes. While bubbling does result in mechanical agitation via induced turbulence in the cuvette, too much bubbling results in sample loss, and electrolysis has been shown to potentially decrease cellular viability.

It was found that high salt/ion content in the media resulted in increased conductivity, elevated temperature (presumably due to Joule heating) and pronounced bubbling, causing low sample recovery and cell death in viability flow cytometry studies with MDA-MB-231 cells. In contrast, pure water solutions exhibited none of these deleterious effects. However, placing cells into hypotonic environments for prolonged periods is known to lead to bursting of cells by osmotic pressure. Despite this, experiments showed that brief (<5 minute) treatment times in ultra-pure water did not significantly reduce cell viability. Furthermore, long time courses of more than 15 minutes could be supplemented with 300 mM sucrose to maintain osmolarity and prevent cellular bursting and viability loss. Ultimately, water and sucrose supplemented water were seen to be more effective candidates for the dissociation of tissues, liquid sample recovery, and preservation of cellular viability, so long as the cellular samples were rapidly removed and immersed in an isotonic PBS solution for further analysis.

Experimental Tissue Dissociation Results

Effect of DC Electric Fields on Tissue Dissociation

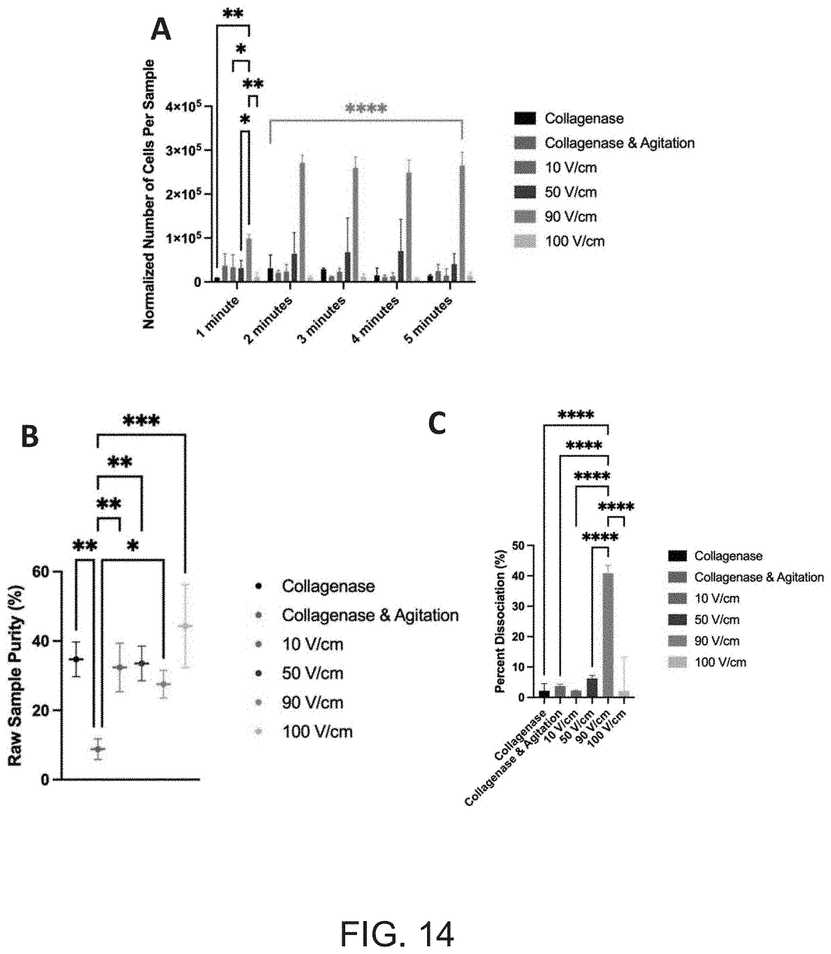

The efficacy of electrical dissociation of tissue into single cells was first assessed at lower-level electric field strengths between 10-100 V/cm over a 5-minute time course using bovine liver tissue in the first electrical setup (FIG. 6). Various metrics were applied to determine a comprehensive understanding of electrical dissociation across different conditions. First, the raw numbers of single and aggregated target tissue cells were retrieved, as measured via flow cytometry (FIG. 14, element A). In FIG. 14, element A, a raw number of cells were processed in a given sample via flow cytometry across various DC electric field conditions as well as control collagenase and collagenase with mechanical agitation conditions in a 5-minute time-course. 90 V/cm trials were significantly more effective at dissociating tissue across 2-5 minute timepoints (p<0.001). Sample purity was assessed by looking at the number of tissue cells with respect to all other particles in suspension, including things like extracellular matrix fragments at 5 minutes (FIG. 14, element B). Finally, this data was used to determine the percent dissociation using the bovine liver tissue compositional model (FIG. 14, element C—Percent dissociation of tissues at 5 minutes. 90 V/cm trials were significantly more effective at dissociating tissues (p<0.001). All quantitative results were collected using flow cytometry.) Cellular debris in the sample was excluded from the analysis. In FIG. 14, elements A-C, one-way ANOVA with Tukey post-hoc analysis and a 95% confidence interval was performed for samples, in element A across a time course and, in elements B and C, at the 5-minute time point. N≥10, * p<0.05, ** p<0.01, ***p<0.001, **** p<0.0001. Information on nonsignificant results is not illustrated on the graph. Colored bars and asterisks represent significance trends across numerous timepoints for a given electrical condition when compared to all other electrical conditions. All significances between 100 V/cm 1 kHz and all other treatments are **** p<0.001 from 2-5 minutes.

In the initial short (<5-minute) time course, maximum percentage dissociation was obtained at 41±3% for applied electric field strengths of 90 V/cm. While there was some variability across time, similar recovery was obtained even after a short duration of 2 minutes in higher applied field strength trials (e.g., 90 V/cm). It is also notable that dissociation was most effective in the 100 V/cm samples, but samples tended to bubble over at E-field strengths of 100 V/cm with non-oscillating voltage in the first experimental setup, which significantly reduced cellular recovery.

Despite reduced cellular recovery due to bubbling in the 100 V/cm condition, this electric field strength afforded the highest sample purity of 44±12%. Sample purity represents a metric of the ratio of single cells to total particles, including cellular aggregates, fragments, debris, and other constituents. This was notably significantly higher than that of the collagenase and mechanical agitation treatment, which surprisingly had the lowest purity of all tested samples at 9±3%, likely due to the presence of ECM fragments or cellular lysis resulting from chemical or mechanical damage.

In order to determine if increase in treatment time can afford an increase in cellular recovery from tissue cores, dissociation efficacy was examined again at field strengths from 10-100 V/cm, using a constant non-oscillating voltage, but over a longer (<30 minute) time course at intervals of 5, 15, and 30 minutes. Representative E field strengths of 10, 50 and 100 V/cm were studied in this manner. Additionally, the second electrical setup was used for long time-course trials to facilitate hands-off processing (FIG. 7).

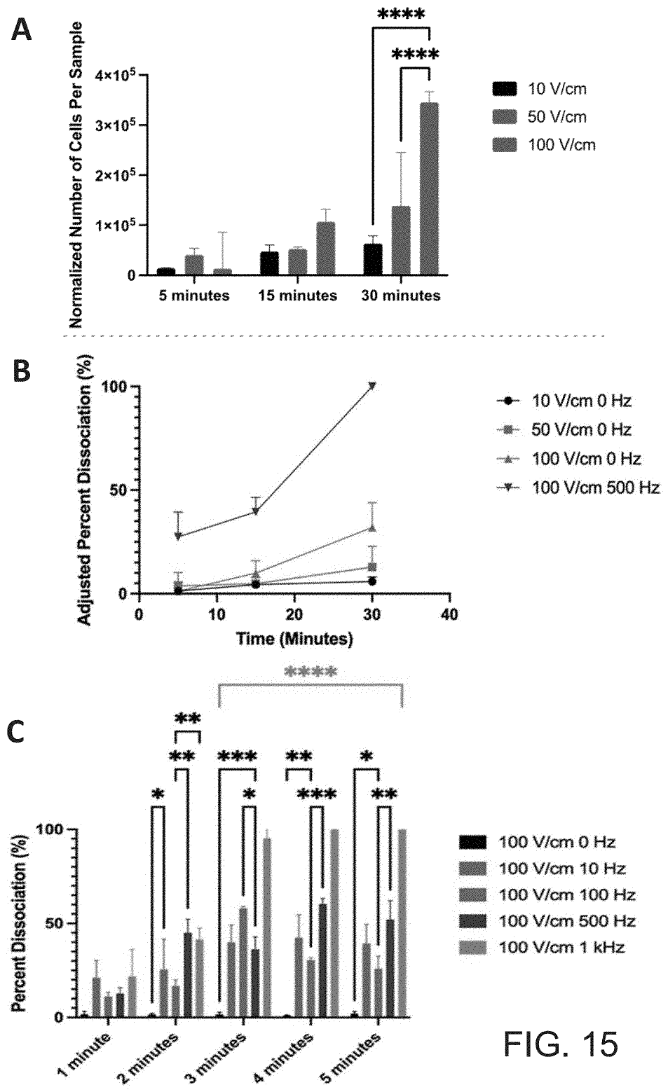

The dissociation results at these constant non-oscillating voltages were quite mild, even over a longer period of 30 minutes −32±12% dissociation was observed after 30 minutes in the 100 V/cm DC treatment, with reduced cellular recovery due to bubble formation again observed in both the 50 V/cm and 100 V/cm trials (FIG. 15, element A—Raw number of tissue cells isolated with constant DC electric fields at a long time course of <30 minutes). This suggests that longer treatments with constant E-fields is not an efficient processing strategy to improve tissue dissociation compared to treatments of shorter length.

Effect of Square-Wave Oscillation on Tissue Dissociation

Subsequently, oscillating voltage was tested as a method to reduce bubble formation and improve processing speed and recovery. The use of oscillating square wave voltages with varying frequencies was found to not only reduce the formation of bubbles, but to significantly improve tissue dissociation across all time points from 2 minutes onward. In a long-term time-course, significantly more cells were recovered at 5, 15 and 30-minute timepoints when applying a 100 V/cm electric field at an intermediate 500 Hz frequency of oscillation while using a square wave function generator, roughly 91±9% in comparison to 32±12% (FIG. 15, element B—Represents percent dissociation with and without oscillating voltage over a long time-course of <30 minutes.). Highly effective dissociation of the tissue was therefore observed after 30 minutes had elapsed, with excellent cellular recovery. These results suggest that oscillating voltage may be a promising candidate for further investigation.

In order to assess the effect of oscillating voltage more thoroughly on cellular dissociation and optimize the process with regards to time, more trials were then conducted on shorter 1-5 minute time points across a range of frequencies of oscillation (FIG. 15, element C, which represents a comparison of normalized percent dissociation in oscillating voltage trials over a short time-course of (<5 minutes). Within these trials, the applied electric field was held constant at 100 V/cm, while the frequency of oscillation was changed. Only square wave functions were used. Two-way ANOVA with Tukey post-hoc analysis and a 95% confidence interval was performed. N≥10, * p<0.05, ** p<0.01, ***p<0.001, **** p<0.0001. Colored bars and asterisks represent significance trends across numerous timepoints for a given electrical condition when compared to all other electrical conditions. Unless otherwise denoted, significance between 100 V/cm 0 Hz treatments and all other treatments is **** p<0.0001 from 2-5 minutes. All significances between 100 V/cm 1 kHz and all other treatments are **** p<0.001 from 2-5 minutes.

500 Hz showed similar results to the 15-minute trial after as little as 2 minutes when switching back to the first device configuration (FIG. 15, elements B and C). Notably, lower frequencies tended to produce less optimal results, consistent with the 100 V/cm results seen in non-oscillating voltage trials (FIG. 13, element A). 1 kHz frequency produced excellent dissociation in a rapid timeframe of <5 minutes, with 95±4% dissociation observed at 3 minutes.

In the 100 V/cm 1 kHz condition, the bovine liver tissue section dissociated completely into a cellular suspension within 4 minutes, which was immediately apparent by visual inspection. Flow cytometry results of dissociated cellular suspensions from the 1 kHz treatment showed excellent cellular recovery and no significant observed cellular fragmentation when examining the size gated results.

While the 30-minute 500 Hz treatment on the second device and 5-minute 1 kHz trial on the first device had similar dissociation efficacy, the lower time requirement of the 1 kHz trial is better suited to clinical translation of the electric field dissociation method. Furthermore, these results were comparable to a 15-minute 1% collagenase/hyaluronidase and optimized mechanical plate shaking trial that was previously characterized as a best chemical/mechanical hybrid condition.

Effect of Tested Electric Fields on Cell Viability and Morphology

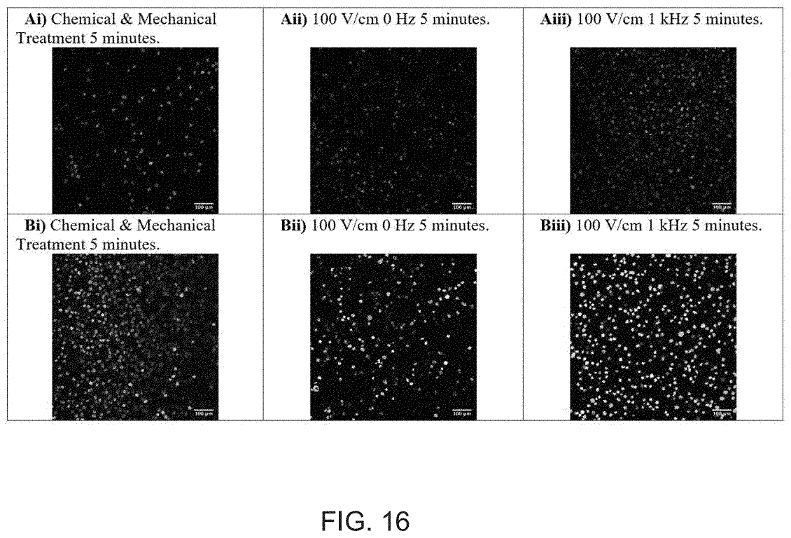

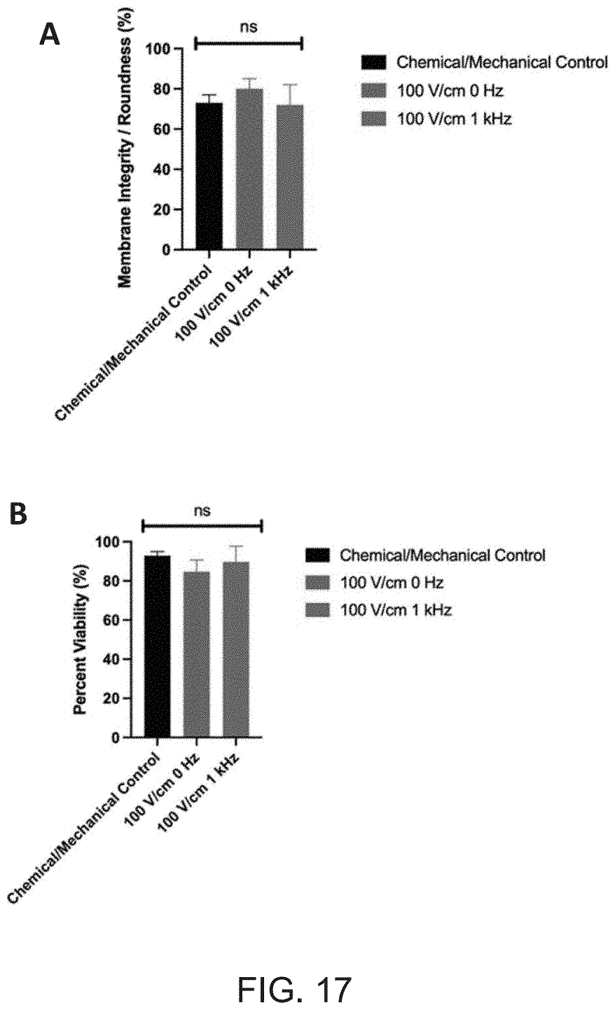

In order to assess whether the electrical treatments significantly affect viability and morphology of cells, live MDA-MB-231 cells were exposed to the same electric fields as the biopsy specimens, and subsequently observed via confocal fluorescence microscopy. There was no statistically significant change in morphology (FIG. 16, elements A sub i-sub iii and FIG. 17, element A) or decline in viability (FIG. 16, elements B sub i-sub iii and FIG. 17, element B) observed, an indication that the low-level electric field treatment did not have a damaging effect on cells. All viability values were 85%—the chemical and mechanical control treatment produced 93±2% viability, 100 V 0 Hz treatment produced 85±6%, and 100 V 1 kHz produced 90±8%. Their morphological roundness percentages were 73±4%, 80±5%, and 72±10%, respectively—in line with what would be expected for this cell type. However, significantly more single cells were observed to have been recovered from electrical treatments after 5 minutes compared to chemical and mechanical treatments. In chemical-mechanical treatments, less cells were recovered and there were more remaining aggregates.

FIG. 16, element A shows morphology images taken with a membrane permeable cell stain, Hoechst 33342. FIG. 17, element B shows live/dead images taken with Hoechst 33342 as well as a membrane-impermeable dead cell stain, DRAQ7. FIG. 16, element A sub i and element B sub i represent samples subjected to the control chemical and mechanical treatment for 5 minutes. FIG. 16, element A sub ii and element B sub ii represent samples subjected to 100 V/cm 0 Hz treatment for 5 minutes. FIG. 16, element A sub iii and element B sub iii represent samples subjected to 100 V/cm 1 kHz treatment for 5 minutes. FIG. 17, element A represents extracted data analysis from images, comparing the best electrical condition to the control chemical and mechanical condition in order to assess morphology (p=0.0755 for the control vs. 0 Hz condition, p=0.9432 for the control vs. 1 kHz condition). FIG. 17, element B uses the same approach to assess viability (p=0.1547 for the control vs. 0 Hz condition, p=0.4520 for the control vs. 1 kHz condition).

Effect of Electric Fields on Mitosis and Cell Cycle Progression

In order to assess whether a 100 V/cm 1 kHz electrical treatment disturbs cell cycle progression at mitosis, a conventional phosphorylated Histone H3 assay was conducted using a AlexaFluor488 conjugated antibody indicative of cells in mitosis. The assay was tested using live, fully passaged MDA-MB-231 cellular suspensions divided into two groups—untreated control, and 100 V/cm 1 kHz treated cells, both with 5-minute trials.

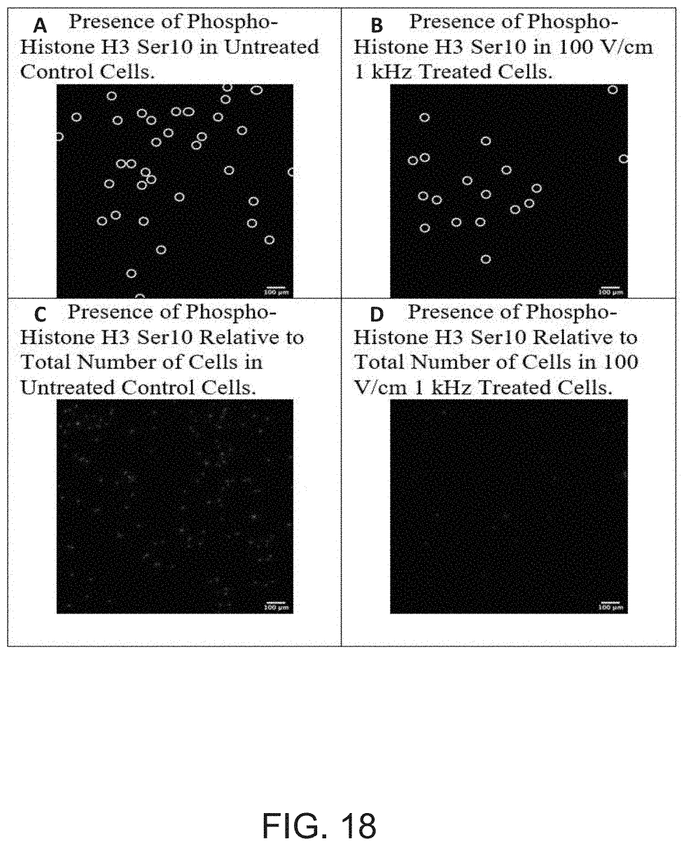

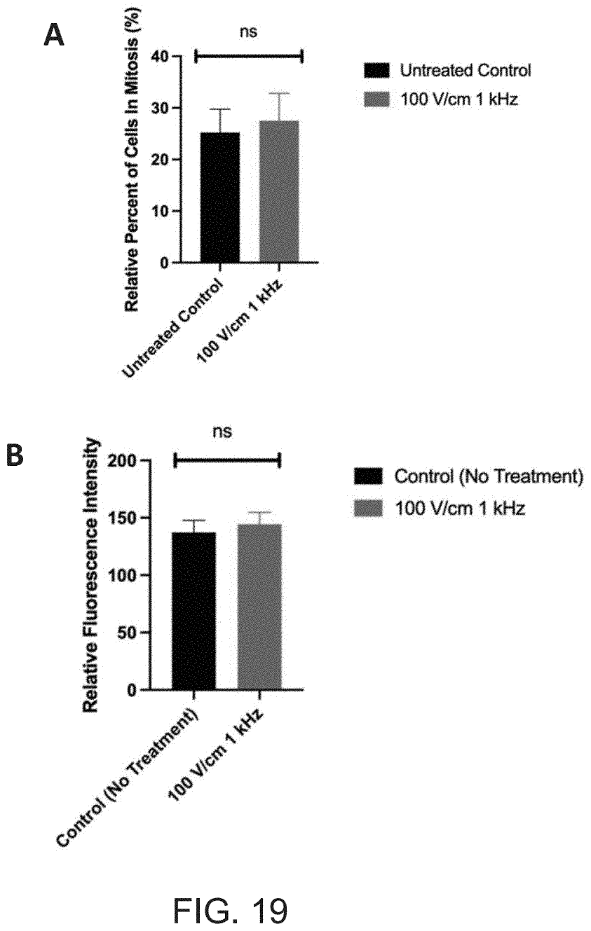

The cells were then observed via confocal fluorescence microscopy. Images for Phospho Histone H3 Ser10 cells were collected (FIG. 18, elements A and B) and overlayed with images of all cells in all other phases with a nonspecific stain (FIG. 18, elements C and D). FIG. 18, elements A and C are representative images for untreated control cells and FIG. 18, elements B and D are representative images of cells treated with the best electrical condition −100 V/cm 1 kHz. After ImageJ processing and statistical analysis with Welch's T-test, it was found that there was no statistically significant change in percent of cells in mitosis or observable effect on progression through the cell cycle (FIG. 19, element A). While untreated controls had 25±4% of cells in mitosis, treated controls had 27±5%. The test was found to be insignificant with a p-value of 0.5464. Spectrofluorometer RFIs were consistent (FIG. 19, element B).

Effect of Electric Fields on cfDNA Release

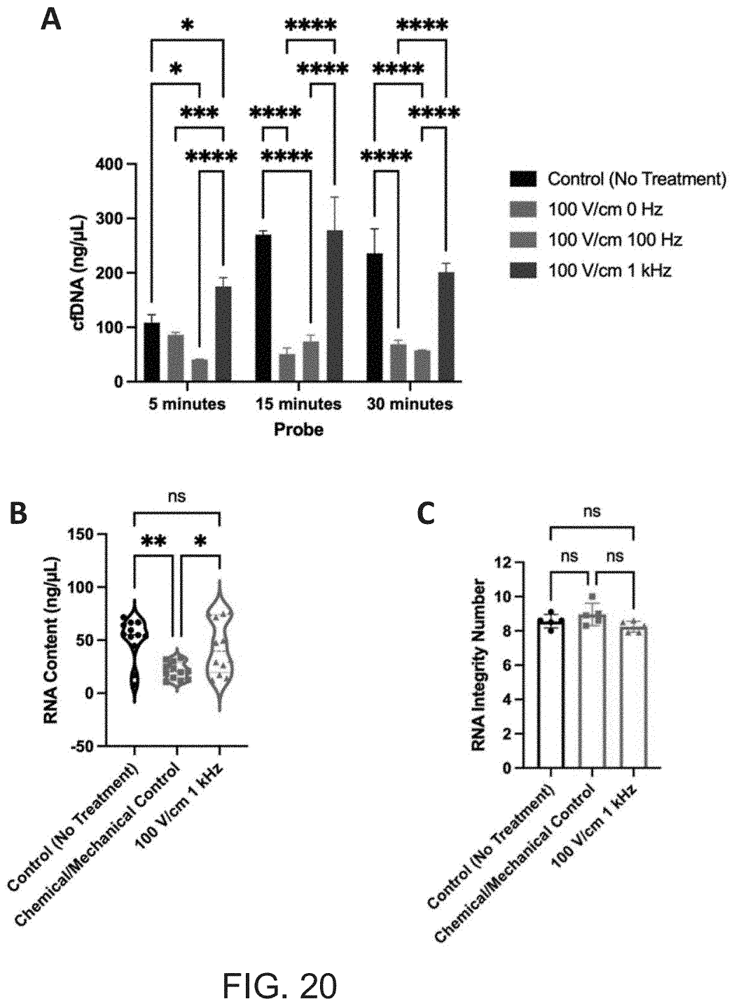

cfDNA release was examined across 5-, 15- and 30-minute timepoints in an untreated control, as well as 100 V/cm electrical treatments at 0 Hz, 100 Hz and 1 kHz. It was found that the electrically treated conditions do not increase the concentration of cfDNA (FIG. 20, element A, showing results for cfDNA in solution in ng/μL from whole tissue section after 5, 15, and 30 minutes of treatment with 100 V/cm at 0 Hz, 100 Hz and 1 kHz, or no treatment. Two-way ANOVA with Tukey post-hoc analysis and a 95% confidence interval was performed). From this preliminary data, it does not appear that cells leak their intracellular contents during processing, a necessary condition to translating this technology to SCS. Interestingly, an observed decrease in cfDNA content in the 0 Hz and 100 Hz conditions may indicate that cfDNA is disrupted at these electric field strengths and oscillation frequencies.

Effect of Electric Fields on RNA and Expression

The original, unadjusted RNA content was not significantly different in the 100 V/cm 1 kHz electrical treatment when compared to the control (FIG. 20, element B, showing results for RNA content in ng/μL after RNA extraction from a starting population of 500,000 cells exposed to no treatment, a collagenase and mechanical agitation treatment, or the 100 V/cm 1 kHz treatment. One-way ANOVA with Tukey post-hoc analysis and a 95% confidence interval was performed (p=0.0014). However, the chemical/mechanical treatment had a slightly lower RNA content when compared to both other groups. All RIN values were 8 or above, consistent with intact RNA (FIG. 20, element C showing results for RNA Integrity Number (RIN) for the extracted RNA samples. One-way ANOVA with Tukey post-hoc analysis and a 95% confidence interval was performed (p=0.0941).

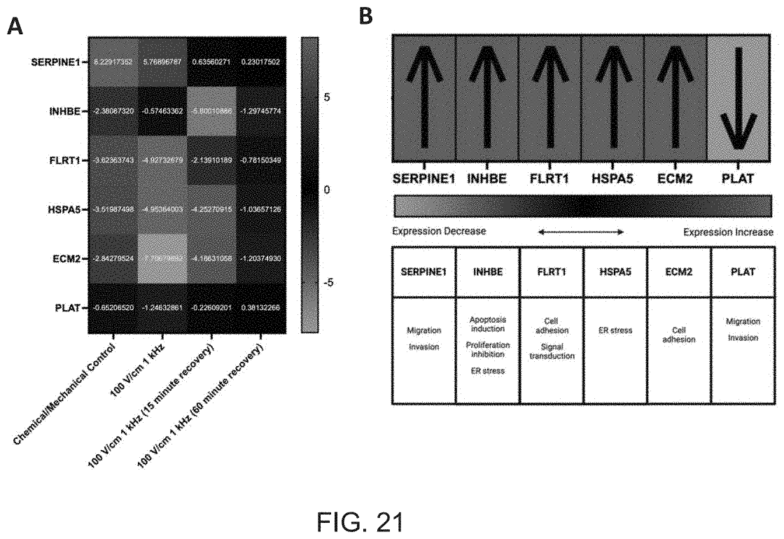

RT-qPCR results showed that a stress response was not observed in the chemical/mechanical or electrically treated cells, apart from the expression of migration and invasion genes SERPINE1 and PLAT, which increased and decreased, respectively (FIG. 21, elements A and B, where element A shows RT-qPCR expression profile changes for 6 indicators across treatments. ACq using a control expression baseline was calculated for each treatment group displayed following best-practices guidelines. Differential expression was then expressed with a heatmap analysis and compared to stress response signatures for the MDA-MB-231 cell line in element B. * p<0.05, ** p<0.01, ***p<0.001, **** p<0.0001). This is consistent with SERPINE1 serving as the principal inhibitor of PLAT, and furthermore an inhibitor of cellular migration.

Notably, FLRT1 and ECM2, both cell adhesion markers, were downregulated in both the chemical/mechanical and electrically treated groups, but more so in the case of the electrical treatment with ACq values of −3.62 vs. −4.93 for FLRT1 and −2.84 vs. −7.71 for ECM2, respectively (FIG. 21, element A). While this is not consistent with established trends of cellular stress in MDA-MB-231, it suggests a potential additional biological mechanism that could enhance tissue dissociation.

15- and 60-minute recovery periods were investigated to determine if these expression trends could be reversed by putting the cells back into their preferred control conditions. Within 60 minutes, the cells can essentially return to their baseline levels, suggesting that the cells will be able to recover characteristic adhesion and migration properties.

Human Glioblastoma Cell Experiments

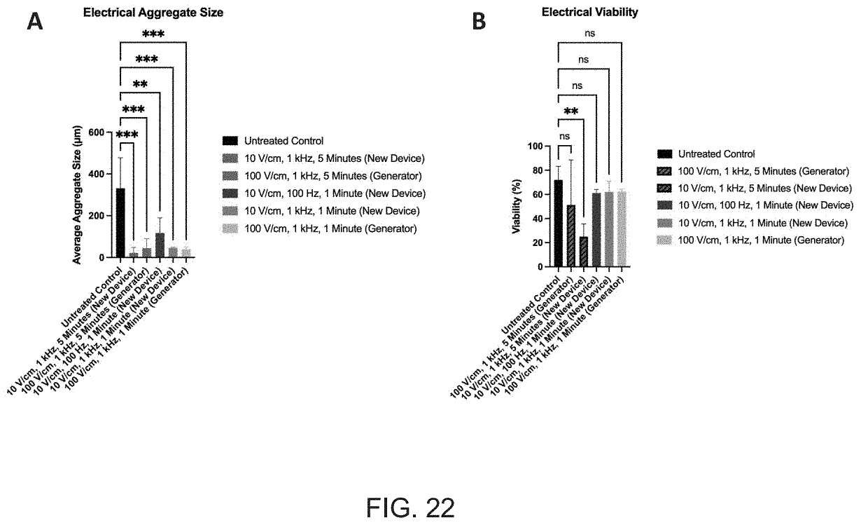

Additional experimental tests were undertaken with human glioblastoma cells to look at clinical translation of the above results. Results of the human clinical glioblastoma tissue tests are shown in FIG. 22, element A (electrical aggregate sizes) and element B (electrical viability). It was found that the electrical aggregate size was significant for all cell separation treatments (statistical significance indicated by ** and *** in A). Electrical viability was found to be statistically significant for a separation under 10 V/cm, 1 KHz, for five minutes using the new device for generation.

From the above description, those skilled in the art will perceive improvements, changes, and modifications. Such improvements, changes and modifications are within the skill of one in the art and are intended to be covered by the appended claims.

1. A system comprising:
a device configured to hold a tissue or cellular sample;
one or more electrodes in contact with the device,
wherein the one or more electrodes are configured to establish an electric field through the device to induce electrical dissociation of the tissue or cellular sample into single cells and/or smaller groups of cells.

2. The system of claim 1, wherein the device is configured to hold the tissue or cellular sample in at least one fluid.

3. The system of claim 2, wherein the at least one fluid comprises a nonionic liquid.

4. The system of claim 3, wherein the nonionic liquid comprises an additive

5. The system of claim 1, wherein the device is configured to hold and process a plurality of tissue and/or cellular samples.

6. The system of claim 1, wherein the device comprises a cuvette, a well, a tube, a microfluidic chip, or another vessel.

7. The system of claim 6, wherein the one or more electrodes are located within the device.

8. The system of claim 6, wherein the one or more electrodes are on located on an outside of the device.

9. The system of claim 1, wherein the electric field through the device behaves as a uniform electric field or a nonuniform electric field in the device.

10. The system of claim 1, wherein a voltage between the one or more electrodes within the device is below an electroporation threshold of the tissue or cellular sample.

11. The system of claim 1, wherein the voltage is an oscillating voltage.

12. The system of claim 1, wherein the electric field is an AC field or a DC field.

13. The system of claim 1, wherein the one or more electrodes of the device are powered through a connection to a power source.

14. A method comprising:
establishing an electric field through a device holding a tissue or cellular sample, wherein the electric field is established by one or more electrodes in contact with the device; and
inducing the tissue or cellular sample to dissociate into single cells and/or smaller groups of cells through electrical dissociation.

15. The method of claim 14, wherein the device is configured to hold and process the tissue or cellular sample in at least one fluid.

16. The method of claim 15, wherein the at least one fluid comprises a nonionic liquid and/or an additive.

17. The method of claim 14, wherein the device is configured to hold and process a plurality of tissue or cellular samples.

18. The method of claim 14, wherein the device comprises a cuvette, a well, a tube, a microfluidic chip, or another vessel.

19. The method of claim 15, wherein the one or more electrodes are located within the device or outside the device.

20. The method of claim 14, wherein a voltage between the one or more electrodes is below an electroporation threshold of the tissue sample.

21. The method of claim 14, wherein a voltage established between the one or more electrodes is an oscillating voltage.
